(19) United States
(12) Patent Application Publication
(10) Pub. No.: US 2026/0151238 A1
(43) Pub. Date: Jun. 4, 2026

(54) DISTRACTING AND ANGLING EXPANDABLE INTERBODY DEVICE

(71) Applicant: Warsaw Orthopedic, Inc., Warsaw, IN (US)

(72) Inventor: Jonathan Dewey, Memphis, TN (US)

(73) Assignee: Warsaw Orthopedic, Inc., Warsaw, IN (US)

(21) Appl. No.: 19/458,999

(22) Filed: Jan. 26, 2026

Related U.S. Application Data
(63) Continuation of application No. 18/651,109, filed on Apr. 30, 2024, now Pat. No. 12,533,241.
(60) Provisional application No. 63/502,471, filed on May 16, 2023.

(51) Int. Cl. A61F 2/44; A61F 2/30

(52) U.S. Cl. CPC A61F 2/4425; A61F 2/447; A61F 2002/30266; A61F 2002/30387; A61F 2002/30398; A61F 2002/30433; A61F 2002/3054; A61F 2002/30579; A61F 2002/443

(57) ABSTRACT

An expandable implant pivotally movable between a collapsed position, an expanded position, and an angled position, is disclosed. The implant may include at least one first pivoting axis extending in a longitudinal direction and at least one second pivoting axis extending in a transverse direction of the implant. A frame may be pivotally coupled to at least one arm, a superior endplate, and an inferior endplate. The at least one arm may be pivotally coupled to one of the superior endplate or the inferior endplate. The implant may include at least one distraction wedge engaged with the at least one arm and at least one angulation wedge engaged with the at least one distraction wedge.

### CROSS-REFERENCE TO RELATED APPLICATIONS

This application is a continuation of U.S. patent application Ser. No. 18/651,109 filed Apr. 30, 2024, which claims the benefit of and priority to U.S. Provisional Patent Application No. 63/502,471 filed May 16, 2023. This application also incorporates by reference: U.S. Pat. No. 11,096,796, titled Interbody spinal implant having a roughened surface topography on one or more internal surfaces, and filed on Mar. 4, 2013; and U.S. Pat. No. 10,821,000, titled Titanium implant surfaces free from alpha case and with enhanced osteoinduction, and filed Jun. 29, 2017. The entire disclosure of each of the above documents is incorporated herein by reference in its entirety.

### FIELD

The present disclosure generally relates to medical devices for the treatment of musculoskeletal disorders, and more particularly to an expandable spinal implant expandable and/or pivotable along multiple different axes, systems for implanting and manipulating the expandable spinal implant, and a method for treating a human spine.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, they may fail to relieve the symptoms associated with these disorders. Surgical treatment of these spinal disorders includes fusion, fixation, correction, discectomy, laminectomy and implantable prosthetics. As part of these surgical treatments, spinal constructs, such as, for example, bone fasteners, spinal rods and interbody devices can be used to provide stability to a treated region. For example, during surgical treatment, interbody devices may be introduced to a space between adjacent vertebral bodies (the interbody space) to properly space the vertebral bodies and provide a receptacle for bone growth promoting materials, e.g., grafting.

More recently, interbody devices have been introduced that provide additional capability beyond static spacing of the vertebral bodies. For example, some devices have expansion capability such that the implant may be introduced to the interbody space in a collapsed state and then expanded to produce additional spacing and, in some cases, introduce or restore curvature to the spine by expanding selectively. However, many existing expandable interbody designs have limited ranges of expansion, limited types of expansion, and limited ranges of inclination.

The present disclosure seeks to address these and other shortcomings in the existing relevant arts.

### SUMMARY

The techniques of this disclosure generally relate to [brief summary of highlights or advantages without tying advantages to a particular example or embodiment].

In one aspect, the present disclosure provides [recitation of claims using ordinary syntax].

In another aspect, the disclosure provides [recitation of additional claimed embodiments as needed]

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIGS. 1A-1B are perspective views of various pivoting axes of an implant in an expanded and lordosed configuration.

FIG. 2 is a side perspective view of the implant in a collapsed configuration.

FIG. 3 is a first perspective view of a frame component of the implant.

FIG. 4 is a second perspective view of a frame component of the implant.

FIG. 5 is a section view of the frame component of FIGS. 3-4.

FIGS. 6A-6B are perspective views of an arm component of the implant.

FIG. 7 is a first partial parts perspective view of the implant with the arm component of FIGS. 6A-6B removed for ease of understanding.

FIG. 8 is a first partial parts perspective view of the implant with the arm component of FIGS. 6A-6B removed for ease of understanding.

FIG. 9 is an exploded parts view of the implant.

FIG. 10 is a partial parts perspective view of the implant with a superior endplate and the arm component of FIGS. 6A-6B removed for ease of understanding.

FIG. 11 is another exploded parts view of the implant.

FIG. 12 is another exploded parts view of the implant.

FIG. 13A is a partial parts exploded view of a first screw component and second screw component.

FIG. 13B is an exploded parts view of the components in FIG. 13A.

FIG. 14 is another partial parts exploded view of the implant.

FIG. 15 is a comprehensive exploded parts view of the implant.

FIG. 16 is a first perspective view of the implant in a distracted configuration.

FIG. 17 is a partial parts perspective view of the configuration shown in FIG. 16 with the arm component removed for ease of understanding.

FIG. 18 is a front view of the implant in a distracted configuration.

FIG. 19 is a partial parts exploded view of select components of the moving mechanism.

FIG. 20A is a partial parts section view of the implant and a driver for distracting the implant.

FIG. 20B is a partial parts section view of the implant of FIG. 20A after the driver has distracted the implant.

FIG. 21A is a partial parts section view of the implant in the collapsed position shown in FIG. 20A.

FIG. 21B is a partial parts section view of the implant in the distracted position shown in FIG. 20B.

FIG. 22A is an alternate partial parts section view of the implant in the collapsed position shown in FIGS. 20A and 21A.

FIG. 22B is an alternate partial parts section view of the implant in the distracted position shown in FIGS. 20B and 21B.

FIG. 23 is a head on partial parts section view of the implant in the distracted position shown in FIGS. 20B, 21B, and 22B.

FIG. 24 is a perspective view of the implant in a lordosed configuration.

FIG. 25 is a partial parts view of the implant in a lordosed configuration with the arm component removed for ease of understanding.

FIG. 26 is a partial partis view of the implant in the lordosed configuration of FIG. 25 with the arm component and frame component removed for ease of understanding.

FIG. 27A is a partial parts section view of the implant in a collapsed position and a driver configured to interact with a second set screw of the moving mechanism.

FIG. 27B is a partial parts section view of the implant of FIG. 20A in an expanded position after the driver has lordosed the implant.

FIG. 28A is a partial parts section view of the implant in the collapsed position of FIG. 27A.

FIG. 28B is a partial parts section view of the implant in the lordosed position of FIG. 27B.

FIG. 29A is an alternate partial parts section view of the implant in the collapsed position of FIGS. 27A and 28A.

FIG. 29B is an alternate partial parts section view of the implant in the lordosed position of FIGS. 27B and 28B.

FIG. 30A is a side section view showing the implant in a collapsed configuration.

FIG. 30B is a side section view showing the implant in a lordosed configuration.

### DETAILED DESCRIPTION

Embodiments of the present disclosure relate generally, for example, to spinal stabilization systems, and more particularly, to surgical instruments for use with spinal stabilization systems. Embodiments of the devices and methods are described below with reference to the Figures.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise.

Various embodiments and components may be coated with a ceramic, titanium, and/or other biocompatible material to provide surface texturing at (a) the macro scale, (b) the micro scale, and/or (c) the nano scale, for example. Similarly, components may undergo a subtractive manufacturing process providing for surface texturing configured to facilitate osseointegration and cellular attachment and osteoblast maturation. Example surface texturing of additive and subtractive manufacturing processes may comprise (a) macro-scale structural features having a maximum peak-to-valley height of about 40 microns to about 500 microns, (b) micro-scale structural features having a maximum peak-to-valley height of about 2 microns to about 40 microns, and/or (c) nano-scale structural features having a maximum peak-to-valley height of about 0.05 microns to about 5 microns. In various embodiments, the three types of structural features may be overlapping with one another, for example. Additionally, such surface texturing may be applied to any surface, e.g., both external exposed facing surfaces of components and internal non exposed surfaces of components. Further discussion regarding relevant surface texturing and coatings is described in, for example, U.S. Pat. No. 11,096,796, titled Interbody spinal implant having a roughened surface topography on one or more internal surfaces, and filed on Mar. 4, 2013—the entire disclosure of which is incorporated herein by reference in its entirety. Accordingly, it shall be understood that any of the described coating and texturing processes of U.S. Pat. No. 11,096,796, may be applied to any component of the various embodiments disclosed herein, e.g., the exposed surfaces and internal surfaces of endplates. Another example technique for manufacturing an orthopedic implant having surfaces with osteoinducting roughness features including micro-scale structures and nano-scale structures is disclosed in U.S. Pat. No. 10,821,000, the entire contents of which are incorporated herein by reference. Additionally, an example of a commercially available product may be the Adaptix™ Interbody System sold by Medtronic Spine and comprising a titanium cage made with Titan nanoLOCK™.

Various embodiments and components of this disclosure may be fabricated from biologically acceptable materials suitable for medical applications, including metals, synthetic polymers, ceramics and bone material and/or their composites. For example, the components, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe, polylactic acid or polylactide and their combinations.

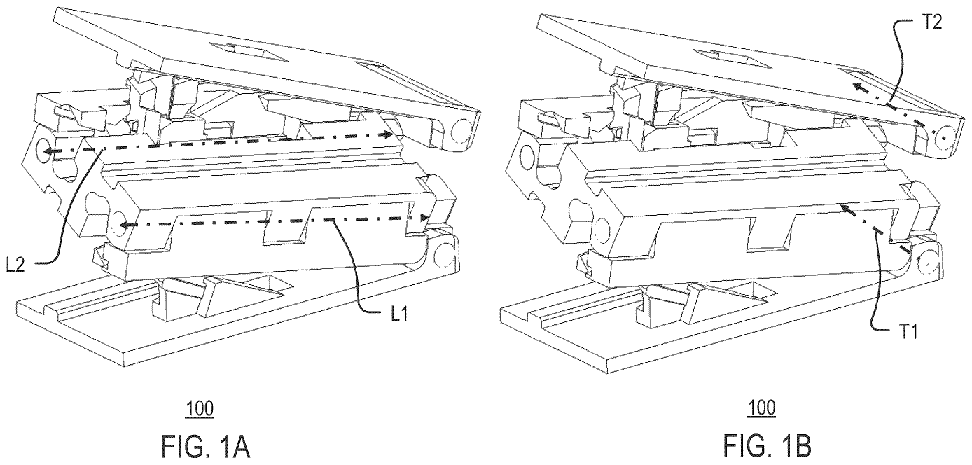

Referring generally to FIGS. 1A-30B an expandable spinal implant 100 and a driver 200 for actuating the implant 100 are shown. Implant 100 may be expanded into a distracted configuration by pivoting about a longitudinal pivoting axis and implant 100 may also be lordosed by pivoting about a transverse pivoting axis. In the example embodiment shown in FIGS. 1A-1B, implant 100 may be distracted by pivoting various internal components about a first longitudinal pivoting axis L1 and a second longitudinal pivoting axis L2 as will be explained in further detail below. Similarly, implant 100 may be lordosed by pivoting various internal components about a first transverse pivoting axis T1 and a second transverse pivoting axis T2 as will be explained in further detail below. In various embodiments, the transverse pivoting axes T1, T2 may be oriented at about 90 degrees (i.e., perpendicularly) with respect to the longitudinal pivoting axes L1, L2. Other relative orientations are contemplated, e.g., +/−about 15 degrees (i.e., about 75 degrees to about 105 degrees).

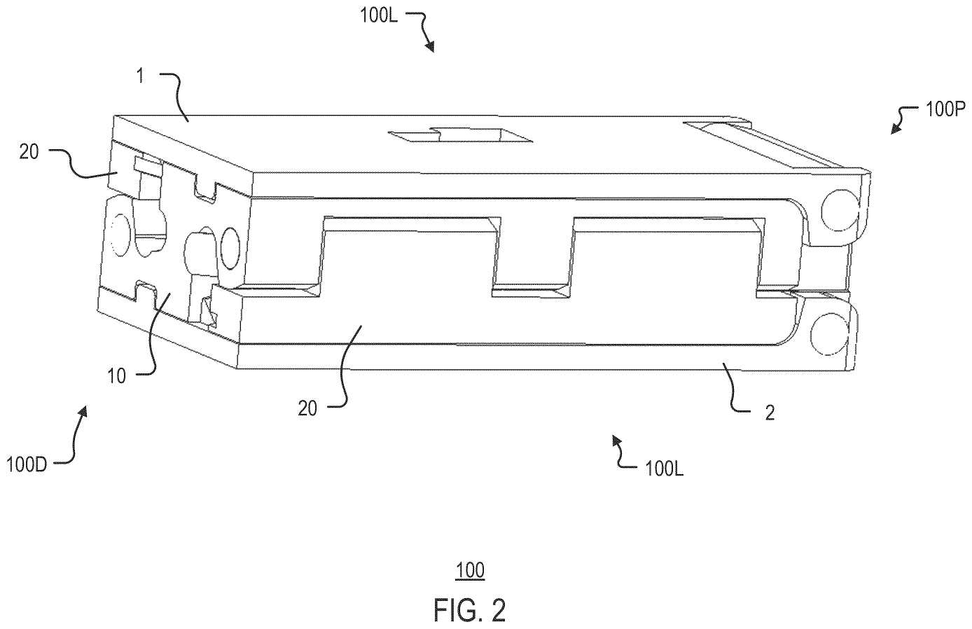

FIG. 2 is a side perspective view of implant 100 in a collapsed configuration. Implant 100 may extend in a lengthwise direction from a proximal end 100P to a distal end 100D. In various embodiments, the lengthwise direction may be parallel with an extension direction of the longitudinal pivoting axes L1 and L2 (at least in the collapsed configuration). Implant 100 may extend in a widthwise direction from a first lateral end 100L to a second lateral end 100L opposite the first lateral end 100L. The widthwise direction may be parallel with an extension direction of the transverse pivoting axes T1 and T2 (at least in the collapsed configuration). A vertical height, or thickness of implant 100, may refer to a distance between an outside surface of the superior endplate 1 to a corresponding outside surface of the inferior endplate 2. As used herein, a “distracted configuration” shall have a meaning consistent with the relevant art field and shall refer to a configuration in which a distance between the superior endplate 1 and the inferior endplate 2 is greater than the same corresponding distance in the collapsed configuration (unless the context clearly indicates otherwise). Similarly, as used herein, an “angled configuration” or “lordosed configuration” shall have a meaning consistent with the relevant art field and shall refer to a configuration in which, from a side perspective view, an interior angle measured between an outside surface of the superior endplate 1 and an outside surface the inferior endplate 2 is greater than the same corresponding interior angle in the collapsed configuration (unless the context clearly indicates otherwise).

As illustrated, implant 100 may include a superior endplate 1 and an inferior endplate 2 that are each pivotally coupled to frame 10 about a respective longitudinal pivoting axis L1, L2. In turn, a first arm 20 and a second arm 20 may each be pivotally coupled to the frame 10 about respective longitudinal pivoting axis L1, L2. Additionally, a first arm 20 may be pivotally coupled to the inferior endplate 2 about transverse pivoting axis T1 and the second arm 20 may be pivotally coupled to the superior endplate 1 about transverse pivoting axis T2 (see FIGS. 1A-1B). Longitudinal pivoting axes L1, L2 may be relied upon for distracting implant 100 and the transverse pivoting axes T1, T2 may be relied upon for angulating the implant 100.

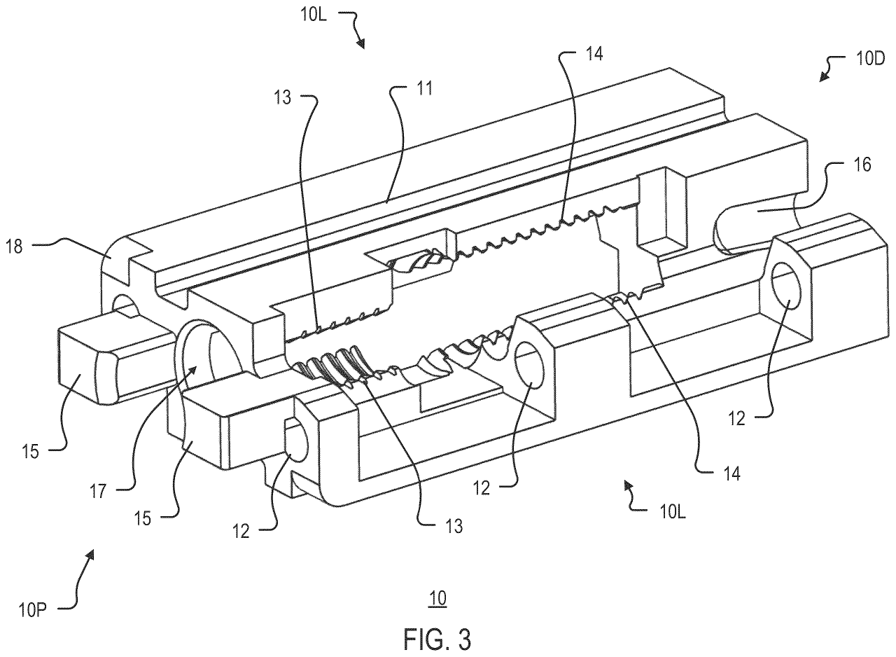

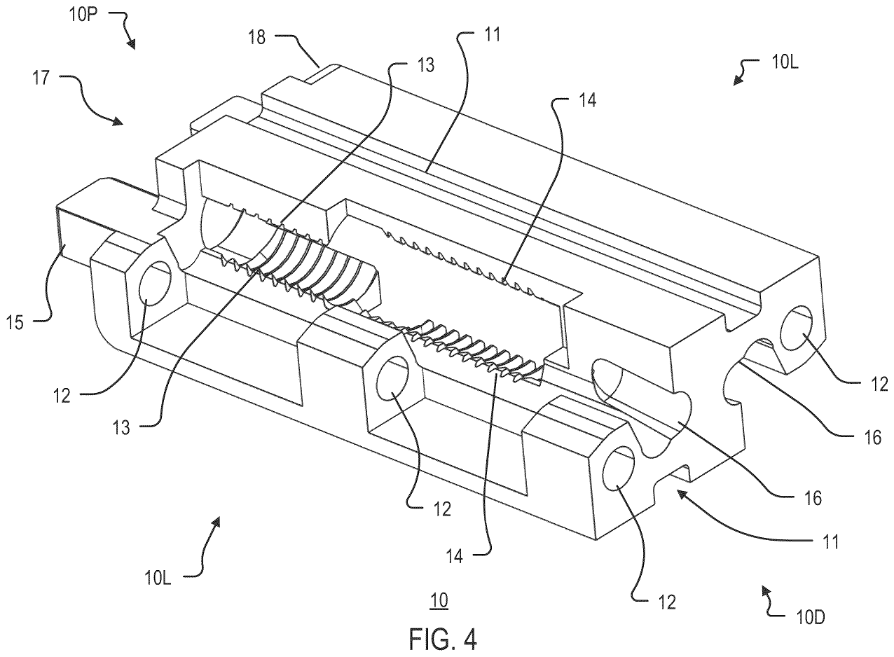

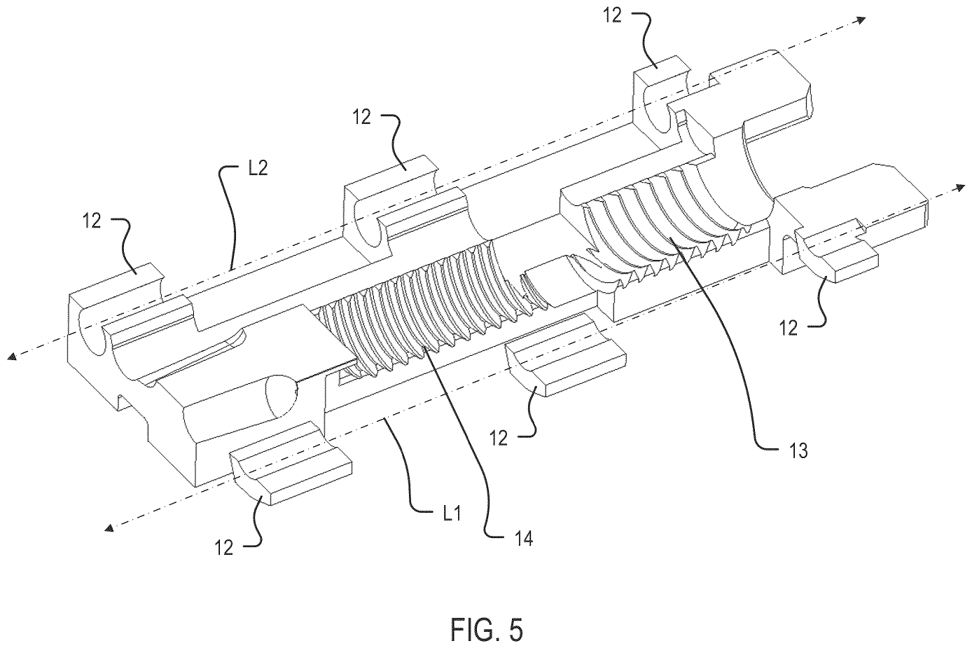

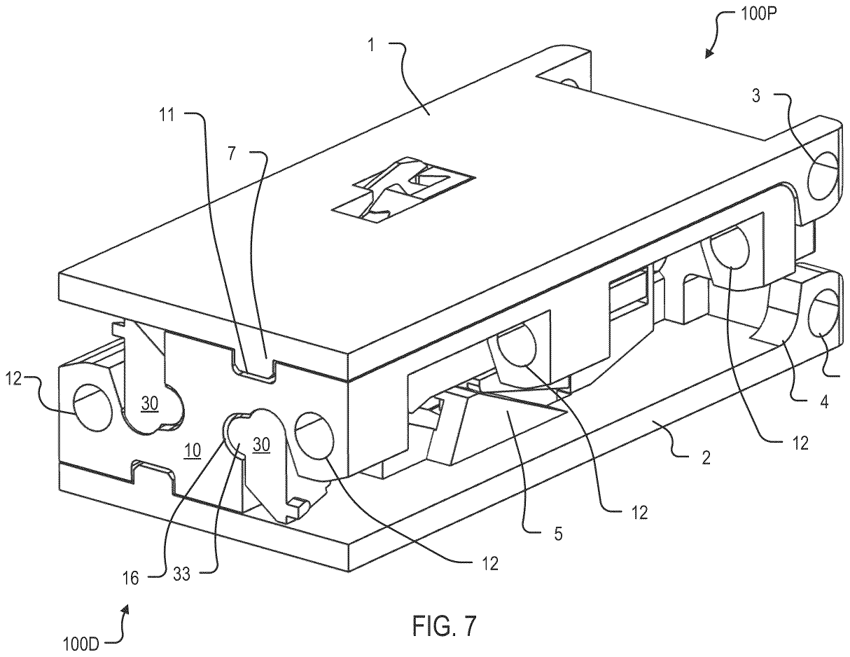

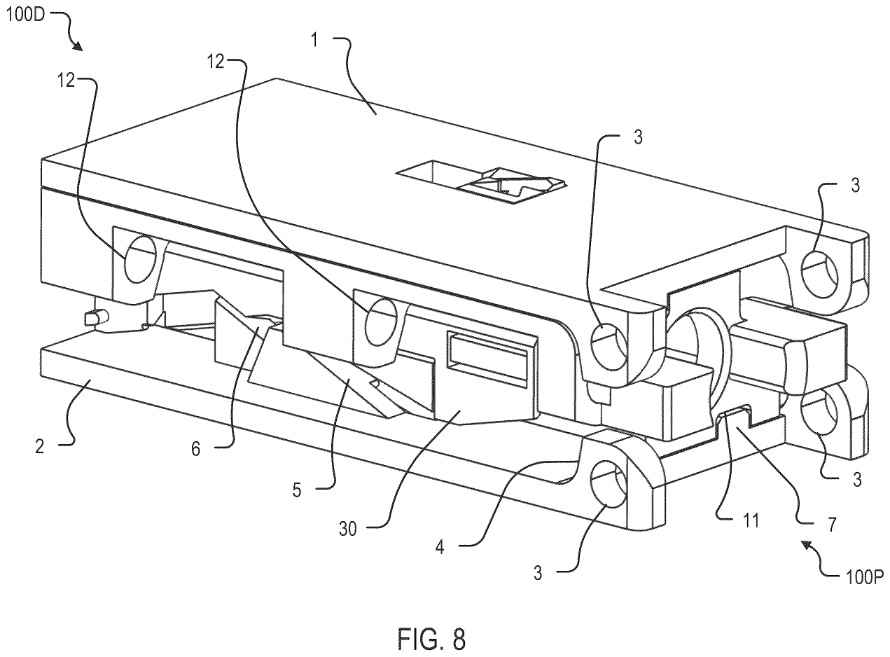

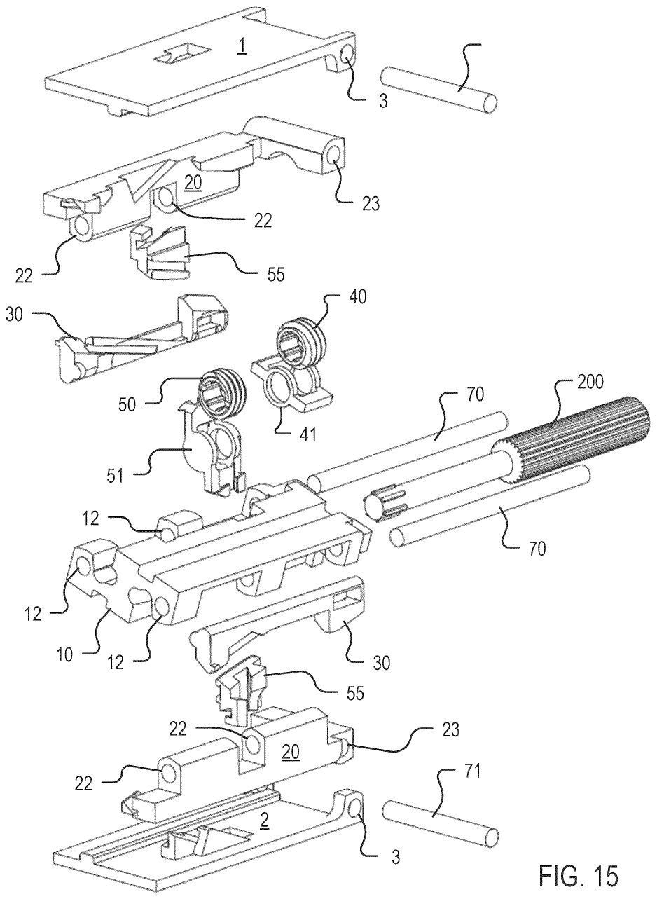

Referring generally to FIGS. 3-5 various views of a frame 10 of an implant 100 are illustrated. FIGS. 3-4 are alternate perspective views of the frame and FIG. 5 is a section view of the frame 10. In various embodiments, frame 10 may be a central support structure which supports or carries various components and allows components to be pivotally coupled thereto. Frame 10 may include a first plurality of frame barrels 12 adjacent a first lateral edge for supporting a first pin 70 (see FIG. 15) extending therethrough to allow pivoting in a functional way similar to a hinge along longitudinal pivoting axis L1. Likewise, frame 10 may include a second plurality of frame barrels 12 adjacent a second lateral edge for supporting a second pin 71 (see FIG. 15) extending therethrough to allow pivoting in a functional way similar to a hinge along longitudinal pivoting axis L2. Frame 10 may include a chamfered edge 18 that functions as a pivoting fulcrum against a corresponding chamfered pocket 4 of the superior and inferior endplates 1, 2 (see FIGS. 7-8). In various embodiments, edge 18 and chamfered pocket 4 do not necessarily touch one another (although they may).

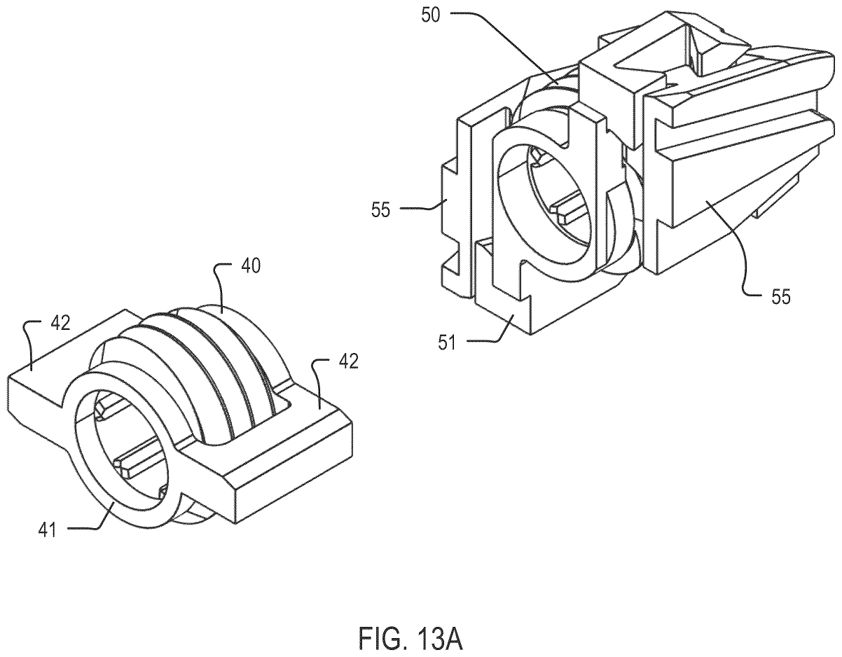

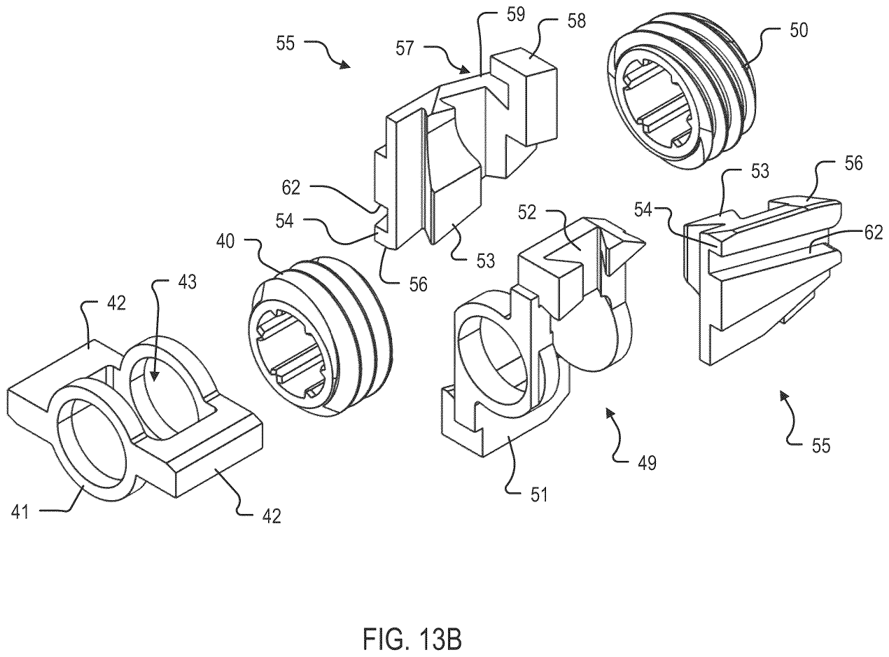

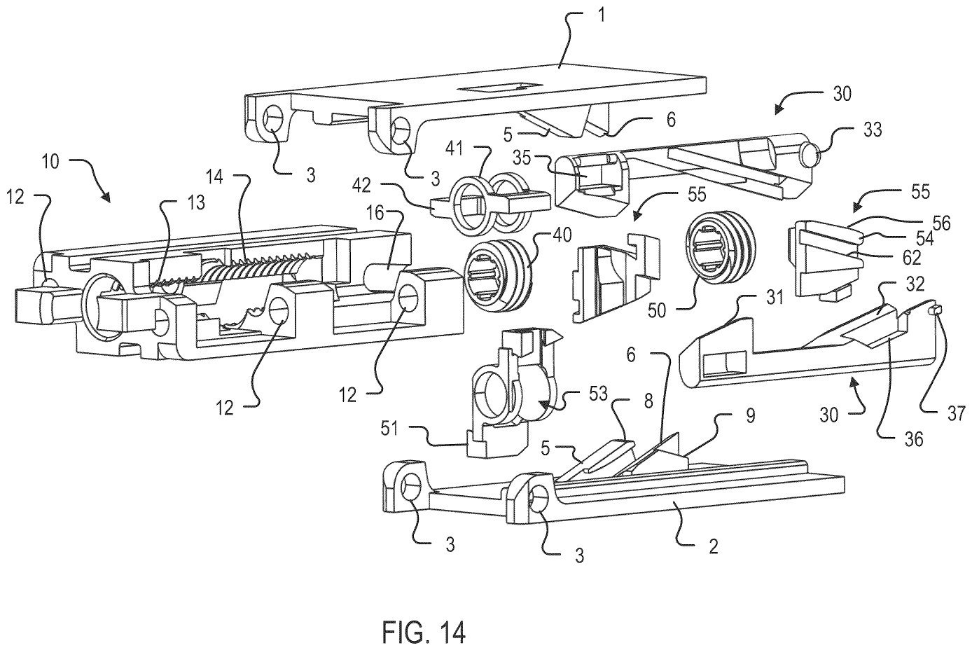

Frame 10 may include a superior channel 11 extending lengthwise down the top surface of frame 10 and an inferior channel 11 extending lengthwise down the bottom surface of frame 10. A corresponding rail 7 of the superior endplate 1 and a corresponding rail of the inferior endplate 2 may be disposable within the corresponding channel 11 (see FIG. 7). Rail 7 may be useful for increasing the structural rigidity of a corresponding endplate 1, 2. Frame 10 may include a first threaded track 13 and a second threaded track 14 that are configured to rotatably support a first set screw 40 and a second set screw 50 (see FIG. 14). In various embodiments, each of the tracks 13, 14 may be coaxially aligned and centered with respect to the lateral side edges 10L of frame 10 and/or the outside surfaces of the superior endplate 1 and inferior endplate 2. Access aperture 17 may be a circular aperture in a proximal side 10P of frame 10 allowing a surgical tool to extend therethrough and rotate either one or both of the first set screw 40 and second set screw 50. Frame 10 may optionally include any type of gripping protrusion 15 on a proximal end 100P thereof for gripping and holding implant 100 during insertion of the implant 100 into a disc space. Frame 10 may also include a superior rotation channel 16 and an inferior rotation channel 16 disposed adjacent a distal side 10D of the frame. Each rotation channel 16 may rotatably support a corresponding ball 33 (or circular outdent) of a corresponding distraction wedge 30 (see FIG. 13) and a bottom cylindrical surface of distraction wedge 30. Generally, geometrical features of the superior side of frame 10 are oriented in a mirror image as to the geometrical features of the inferior side of frame 10. Accordingly, features shown with respect to the superior side of frame 10 shall be understood to be similar, but oppositely oriented with respect to a central axis extending through access aperture, to those of the inferior side of frame 10. This “mirror image” configuration facilitates the distraction and lordosing of implant 100 and is represented in many components of this disclosure.

FIG. 5 is a section view lengthwise through frame 10. In the example illustration, it is shown that three barrels on a first side of frame 10 are configured to rotatably support a first pin (not illustrated) extending therethrough along longitudinal pivoting axis L1 and three barrels on a second side of frame 10 are configured to rotatably support a second pin (not illustrated) extending therethrough along longitudinal pivoting axis L2. Additionally, it is shown how first threaded track 13 and second threaded track 14 are centered with respect to one another.

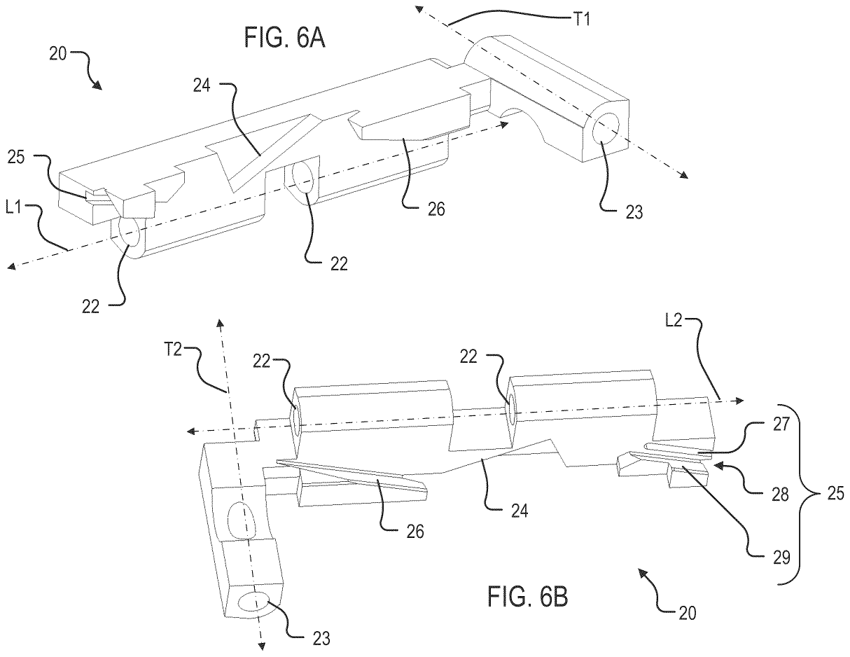

FIGS. 6A-6B are perspective views of an arm 20 of implant 100. Implant 100 may include first and second arms 20. In various embodiments, each arm 20 may extend in a longitudinal direction from a proximal end 20P to a distal end 20D. Additionally, each arm 20 may include an inside side surface 20B that faces inward towards a center of implant 100 and an outside side surface 20A that faces away from a center of implant 100. A first arm 20 may be pivotally coupled to frame 10 on one side thereof and a second arm 20 may be pivotally coupled to frame 10 on an opposite side thereof. For example, first arm 20 may include first and second barrels 22 that are configured to rotatably support a pin 70 (see FIG. 15) extending therethrough along longitudinal pivoting axis L1. Likewise, second arm 20 may include first and second barrels 22 that are configured to rotatably support a pin 70 (see FIG. 15) extending therethrough along longitudinal pivoting axis L2. In this way, the barrels 22 of first arm 20 may be aligned with the frame barrels 12 of frame 10 such that pin 70 extends through barrels 22 and frame barrels 12 thereby defining longitudinal pivoting axis L1 and allowing the first arm 20 to pivot relative to the frame 10. Similarly, the barrels 22 of second arm 20 may be aligned with the frame barrels 12 of frame 10 such that pin 70 extends through barrels 22 and frame barrels 12 thereby defining longitudinal pivoting axis L2 and allowing the second arm 20 to pivot relative to the frame 10. In various embodiments, one arm 20 may pivot in a direction that urges superior endplate 1 away from frame while the other arm 20 may pivot in an opposite direction that urges inferior endplate 2 away from frame 10. Each arm may also include a barrel 23 that comprises an aperture extending therethrough in a direction transverse to the apertures of barrels 22. The barrel 23 of first arm 20 may pivotally support a pin 71 (see FIG. 15) extending therethrough along the transverse pivoting axis T2 that also extends into and/or rotatably couples with a corresponding endplate barrel 3 of the superior endplate 1 (see FIG. 15). Likewise, the barrel 23 of second arm 20 may pivotally support a pin (not illustrated) extending therethrough along the transverse pivoting axis T1 that also extends into and/or rotatably couples with a corresponding endplate barrel 3 of the superior endplate 1 (see FIG. 15).

Each arm 20 may include various pockets, inclined surfaces, ramps, protrusions, indents, and channel like features that allow it to interact with a corresponding arm 20, distraction wedge 30, and angulation wedge 55 (see FIG. 15). For example, the inside side surface 20B of each arm 20 may include a plurality of ramps 26, 24, 25. As used herein, the term ramp shall have a broad meaning describing any surface that is inclined with respect to an adjacent surface and with which another component, e.g., a wedge or rail, may slide over and/or be in sliding contact with. In some cases, a ramp may also have additional geometrical features such as coordinating protrusions, bosses, and/or indentations that may be useful for slidably mating with other components. In this way, a “ramp” may also have channel like attributes and shall not be limited solely to an inclined surface. For example, inside ramps 24 and 26 may take the form of an inclined surface without any coordinating protrusions, indentations, etc. while second ramp 25 may include a sliding surface 29 and a coordinating rail 27 that form a channel 28 therebetween (see lower right hand corner of FIG. 6B). Accordingly, it shall be understood that a “ramp” may comprise any combination of inclined surfaces, rails, and/or channels unless the context clearly indicates otherwise.

FIG. 7 is a partial parts distal perspective view of implant 100 with the arm component 20 removed and FIG. 8 is a partial parts proximal perspective view of implant 100 with the arm component 20 removed for ease of understanding. In FIG. 7, it is shown how the distraction wedges 30 are in contact with the frame 10. For example, the balls 33 of each respective arm are disposed within the corresponding channel 16. FIG. 8 illustrates the orientation of distraction wedge 30 from the proximal end 100P. Additionally, FIGS. 7 and 8 illustrate the ramps 5, 6 along the inside surface of the superior endplate 1 and inferior endplate 2.

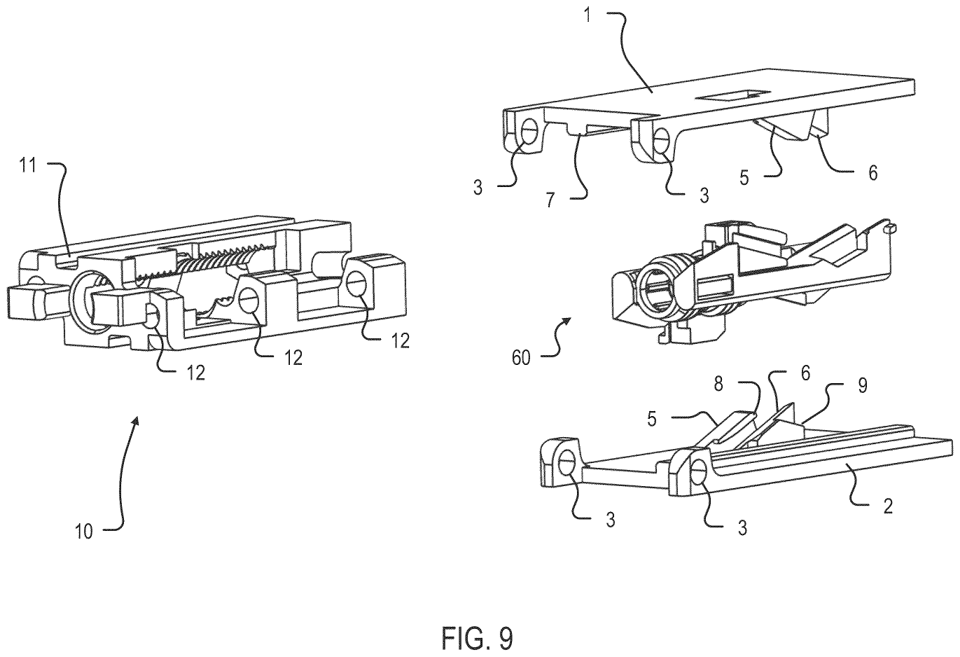

Referring generally to FIGS. 9-15, various exploded parts and partial parts views of implant 100 are illustrated. In each of these views the arms 20 are removed for ease of understanding. In FIG. 9, the superior endplate 1 and inferior endplate 2 are separated away from the frame 10 to expose a moving mechanism 60. Moving mechanism 60 may comprise various distraction wedges 30 and angulation wedges 55 which are each selectively operable by rotating one or both of the first set screw 40 and second set screw 50 as will be explained in more detail below.

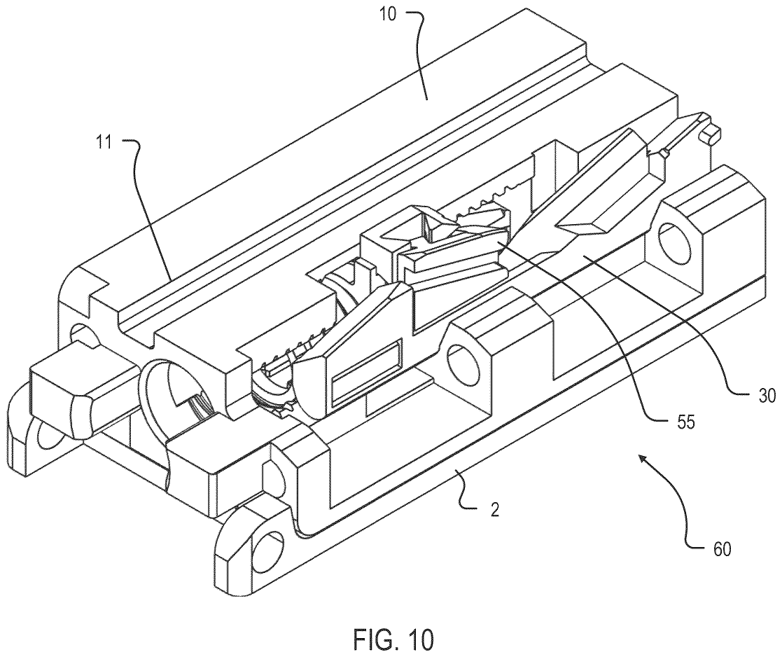

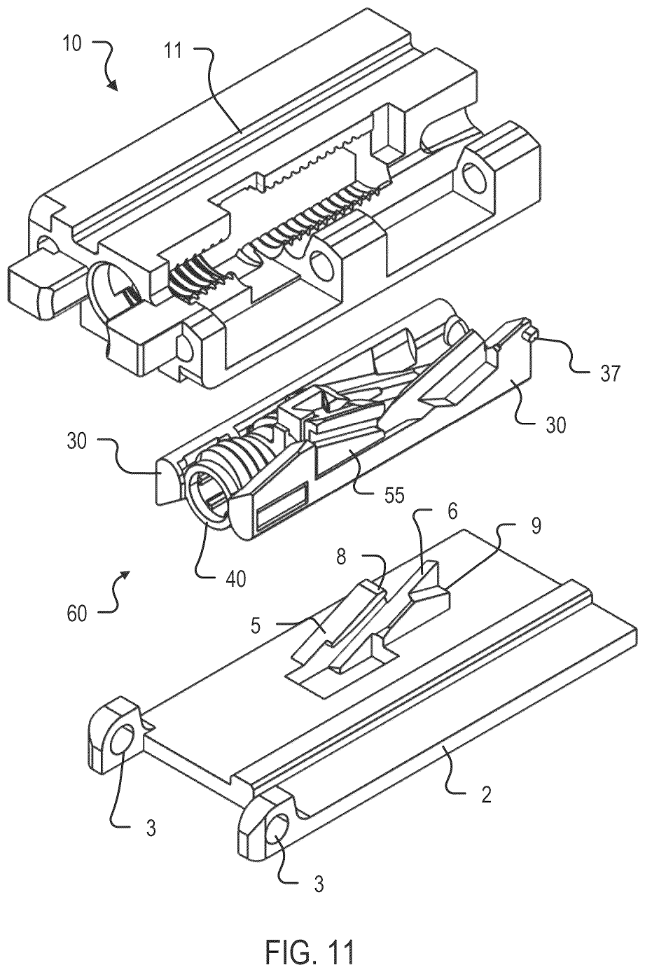

FIG. 10 illustrates implant 100 with the superior endplate 1 and the arm 20 removed for ease of understanding of moving mechanism 60. In the example illustration, it is shown how a first side of moving mechanism 60 includes a first distraction wedge 30 and an angulation wedge 55. FIG. 11 shows the moving mechanism 60 removed from frame 10 and adjacent to inferior endplate 2. In this illustration, it is shown that inferior endplate 2 may include a first ramp 5 and a second ramp 6 that are inclined at the same angle relative to the interior surface of inferior endplate 2. First ramp 5 may include an overhang portion 8 that forms a channel between ramp 5 and ramp 6. Additionally, a third ramp 9 may be inclined on the interior surface of inferior endplate 2. In various embodiments, third ramp 9 may be inclined in an opposite direction compared to ramps 5 and 6 for reasons that will be made apparent below.

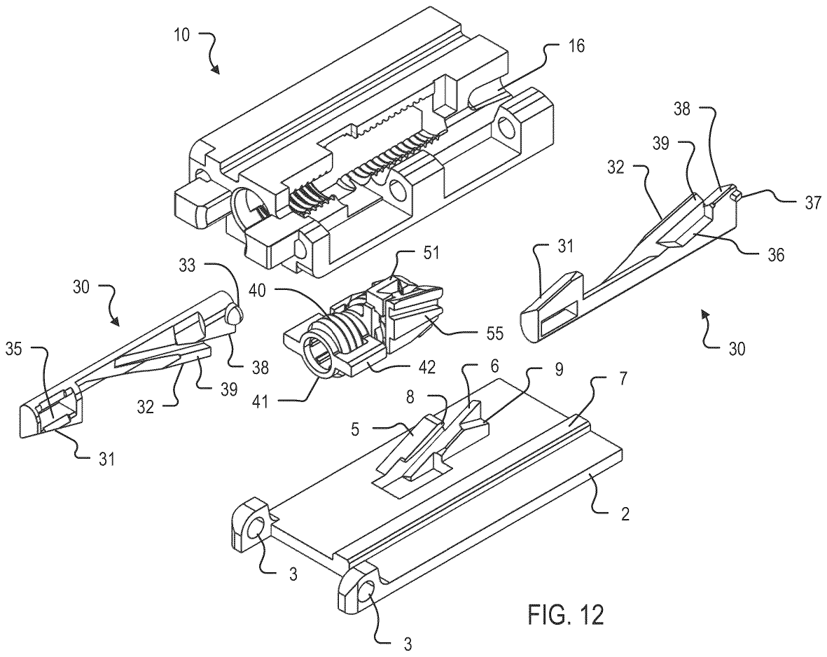

FIG. 12 is another exploded parts view of implant 100 in which the arms 20 have been separated to show the various features of distraction wedge 30 and the other components of moving mechanism 60. For example, in between the distraction wedges 30 a first set screw 40 and a second set screw 50 are operably coupled to various components to cause the distracting and angulating of the implant 100. In FIG. 12, second set screw 50 is not labeled and only partially visible (see FIGS. 13, 14). Distraction wedge 30 may include a plurality of inclined surfaces or ramps 31, 32, 38. Ramp 32 may also protrude from a side of distraction wedge 30 to form rail portion 39 for connecting with a corresponding channel of angulation wedge 55 as will be explained below. Additionally, the outside surface of each distraction wedge 30 that faces the adjacent arm 20 may include at least one inclined protrusion 37 having top and bottom sliding surfaces. The inclined protrusion 37 may be configured for sliding disposal inside a corresponding channel 28 of an arm 20 such that the top and bottom surfaces of protrusion 37 slide along the corresponding surfaces of ramp 25 thereby slidably mating the distraction wedge 30 and arm 20.

FIGS. 13A-13B illustrate a first set screw 40 and a second set screw 50 operably coupled with various components. First set screw 40 may be operably coupled to a first set screw frame 41 having first and second protrusions 42 (also referred to as bosses). In various embodiments, first set screw 40 may be disposed inside of a pocket 43 of first set screw frame 41 such that first set screw 40 may be rotated within track 13 and be linearly translated along a length of track 13. This loose type of coupling arrangement allows frame 41 to rotate as needed as the distraction wedges 30 move forward and backward in the longitudinal direction and up and down in the vertical direction. Additionally, second set screw 50 is operably coupled to a second set screw frame 51. For example, second set screw 50 may be disposed inside of a pocket 49 of frame 51 such that second set screw 50 may be rotated within track 14 and be linearly translated along a length of track 14. This loose type of coupling arrangement also allows frame 51 to rotate as needed as the angulation wedges 55 move forward and backward in the longitudinal direction and up and down in the vertical direction. Second set screw frame 51 is operably coupled to a pair of angulation wedges 55 (see FIG. 13A). Each angulation wedge 55 may include a rail 53 having a size and shape corresponding to a respective channel 52 of the second set screw frame 51. In the example embodiment, channels 52 of frame 51 extend in a vertical direction and therefore allow the angulation wedges 55 to move up and down relative to frame 51 by sliding against corresponding ramps 5, 6 of the superior and inferior endplates 1, 2. Each angulation wedge 55 may include a sliding ramp surface 56 that is configured to slide across the inclined surface of ramp 6 and a sliding surface or ramp 62 that is configured to slide across the inclined surface of ramp 5. Additionally, rail portion 54 of angulation wedge 55 may be disposed in the channel formed between ramps 5 and 6 and overhang portion 8. In this way, each angulation wedge 55 may be operably coupled to and mated with a corresponding endplate 1, 2. Each distraction wedge may also include a C-shaped overhang 58 that hangs over sliding ramp surface 59 and thereby forms a channel 57 therebetween. The rail 39 of distraction wedge 30 may be disposed in the channel 57 and thereby slidably coupled to and mated with the distraction wedge 30 and angulation wedge 55. Stated another way, this arrangement allows components to slide forward and backward with respect to one another without becoming disconnected during operation.

FIG. 14 is another exploded parts view of implant 100 without the arms 20 illustrated for ease of understanding. FIG. 15 is an exploded parts view of implant 100 with all of the components illustrated. In FIG. 15, generally only the primary components and most important features necessary to explain the order of assembly of the implant 100 are labeled because the various sub-features are more easily visible in the other larger exploded parts views (see, e.g., FIG. 9, and 11-14). FIG. 15 illustrates the arms 20 and longitudinal pins 70 that extend along longitudinal axes L1 and L2 (see FIGS. 1A and 5), respectively, as well as the transverse pins 71 that extend along transverse axes T1 and T2 (see FIGS. 1B and 6B), respectively. In the example embodiment it is shown that each of the superior and inferior endplates 1, 2 are operably coupled to a corresponding arm 20 by way of a corresponding transverse pin 71. For example, each transverse pin 71 may extend through the apertures defined by the two corresponding endplate barrels 3 and the aperture defined by the arm barrel 23. Each arm 20 may be operably coupled to the frame 10 by the longitudinal pin 70. For example, each longitudinal pin 70 may extend through the apertures defined by the two arm barrels 22 and the apertures defined by the three frame barrels 12. When assembled, the two arm barrels 22 may be disposed between the three frame barrels 12 in alternating sequence. It shall be understood that the number, size, orientation, and shape, of frame barrels 12 and arm barrels 22, 23 may vary from embodiment to embodiment. As explained previously, the frame 10 may operably support the first set screw 40 and second set screw 50 within the tracks 13, 14 (see FIG. 14). The first set screw 40 may be operably coupled to first set screw frame 41 and the second set screw 50 may be operably coupled to second set screw frame 51. Thus, the first set screw 40 may be operably coupled to the distraction wedges 30 via first set screw frame 41. The second set screw 50 may be operably coupled to the angulation wedges 55 via the second set screw frame 51. Additionally, the distraction wedges 30 may be operably coupled to the angulation wedges 55 as explained previously. Each of the set screws 40, 50 may be rotated by a corresponding surgical tool 200 (e.g., a torx or hexalobe driver) as will be explained in further detail below.

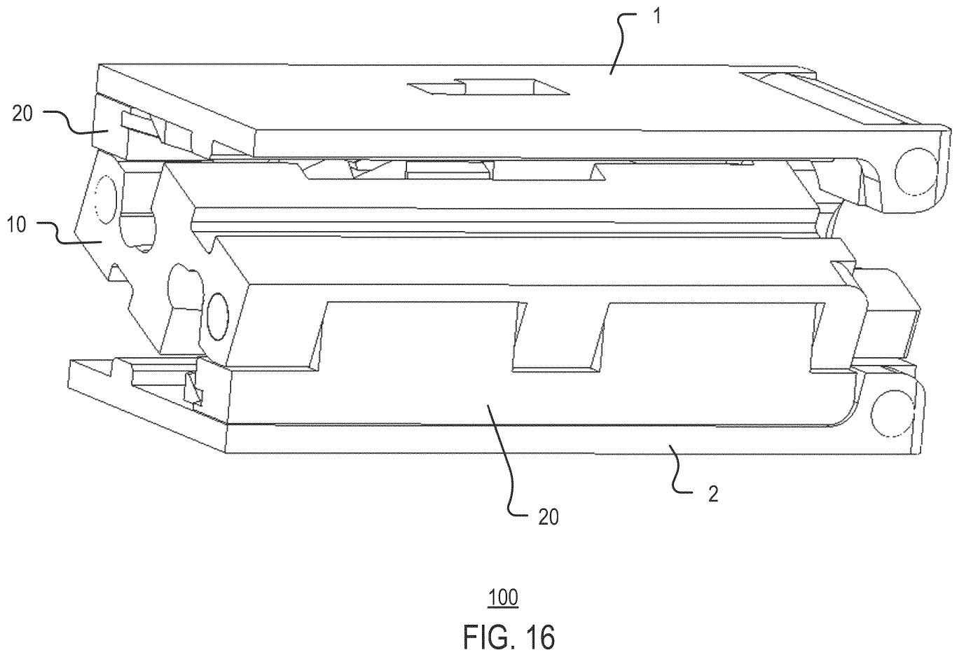

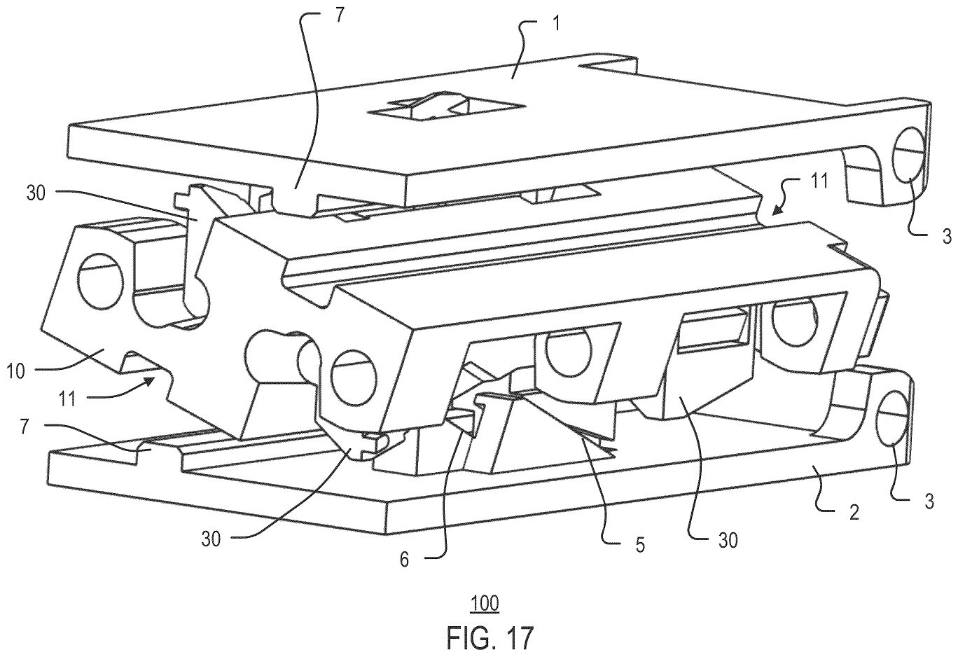

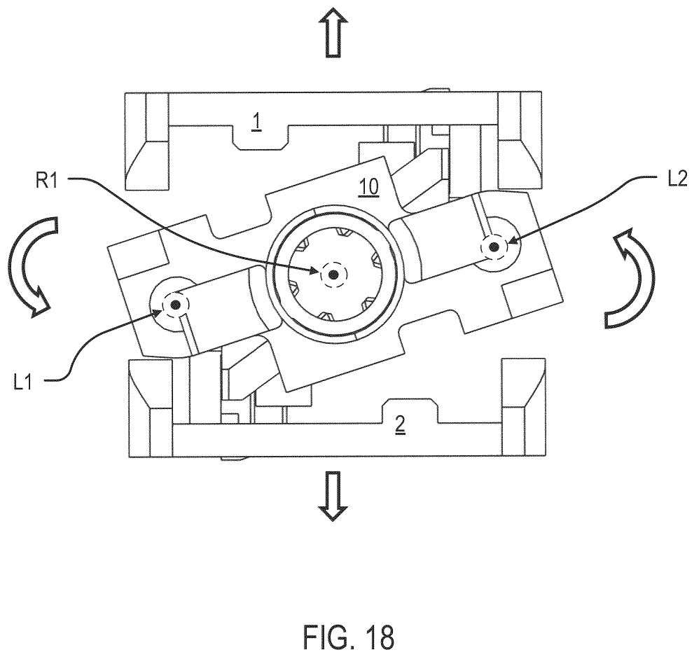

Referring generally to FIGS. 16-18, various views of implant 100 positioned in a distracted position are shown. In the particular configuration of FIG. 16, implant 100 may be considered to be parallel distracted on account of there being no relative change in the angle of inclination between the superior endplate 1 and inferior endplate 2. For example, a distance between the outside surfaces of superior endplate 1 and inferior endplate 2 is greater than in the collapsed configuration and the implant 100 is not lordosed. Distraction of implant 100 may be achieved by action of the first set screw 40 moving the distraction wedges 30 from the distal end 100D of implant 100 towards the proximal end 100P of implant 100. With reference to FIG. 17, the arms 20 are removed to show how the distraction wedges 30 have linearly moved in the longitudinal direction of implant 100 and how the frame 10 may rotate while the cylindrical portion of distraction wedges 30 remain within the circular channel 16 of frame 10. This configuration allows the distraction wedges 30 to move forward and backward while the frame 10 rotates (pivots about L1 and L2). With reference to FIG. 18, first set screw frame 41 may be rotated around rotation axis R1 thereby linearly moving distraction wedges 30 and causing frame 10 to rotate about the rotation axis R1. Rotation axis R1 may extend in a longitudinal direction of implant 100 and be defined by the tracks 13, 14. In the illustrated embodiment, rotation axis R1 may extend through the center of frame 10 and implant 100 in the longitudinal direction. In other embodiments, R1 may be offset from center of frame 10 and implant 100. As illustrated, due to the way that superior endplate 1 is coupled to frame 10 by longitudinal pin 70 the superior endplate 1 is urged upward in the vertical direction by pivoting along longitudinal pivoting axis L2. Similarly, due to the way that inferior endplate 2 is coupled to frame 10 by longitudinal pin 70 the inferior endplate 2 is urged downward in the vertical direction by pivoting along longitudinal pivoting axis L1.

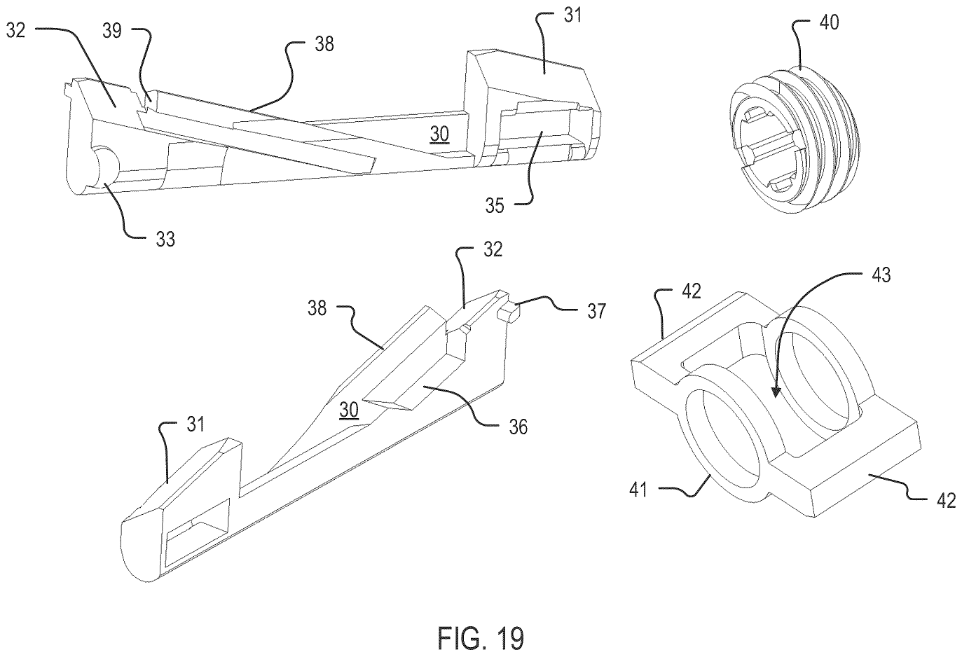

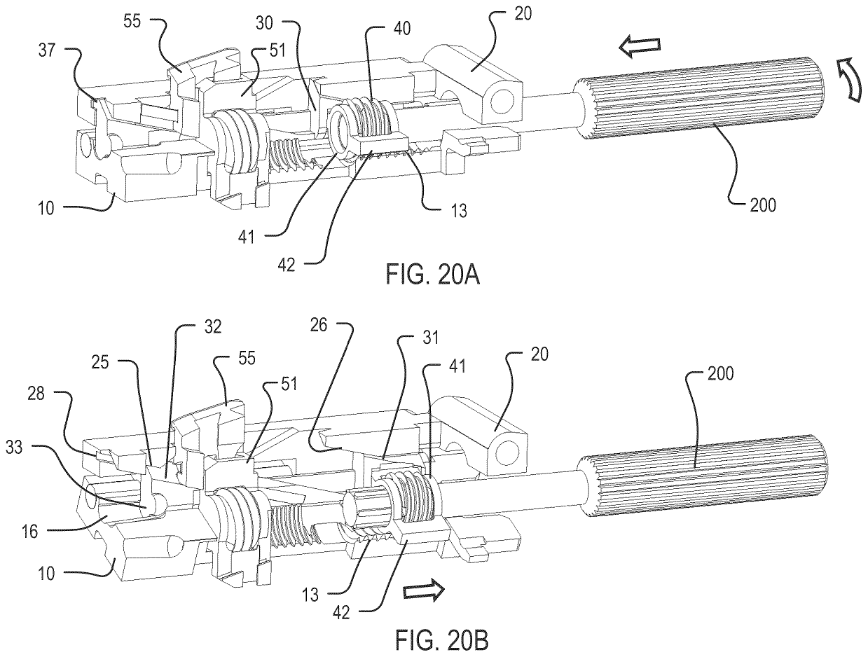

Referring generally to FIG. 19 and FIGS. 20A-23 various views showing the components responsible for distraction of implant 100 are illustrated. Briefly recall, first set screw 40 may be disposed in the pocket 43 of first set screw frame 41. Each wing like protrusion 42 may be disposed within a corresponding cavity 35 (also referred to as an eyelet) of one of the distraction wedges 30. For example, the protrusions 42 are nested within cavity 35 but are not directly coupled by a rigid connection. Ramps 31 and 32 may act against corresponding ramps 25, 26 of arm 20 to thereby wedge apart arm 20 (i.e., drive it apart). Additionally, protrusion 37 may be mated with arm 20 via channel 28 (see FIG. 6B). Furthermore, rail 39 may be mated with the angulation wedge 55. In the collapsed position, angulation wedge 55 may be nested within relief area 36 (see FIG. 12). In this embodiment, movement of solely the distraction wedge 30 by only actuating first set screw 40 will not cause the angulation wedges 55 to adjust the angulation of the superior and inferior endplates 1, 2. However, the angulation wedges 55 may move upward and downward, respectively, on account of being mated to the corresponding endplate 1, 2 and the distraction wedge 30 (see FIG. 20). Stated another way, each angulation wedge 55 may move up or down in the vertical direction with respect to the second set screw frame 51 as distraction wedge 30 is linearly translated (see FIG. 20B) and the angulation wedge 55 maintains its same relative position with respect to arm 20. In at least some embodiments, second set screw frame 51 may rotate or tilt about its rotation axis to accommodate this functionality.

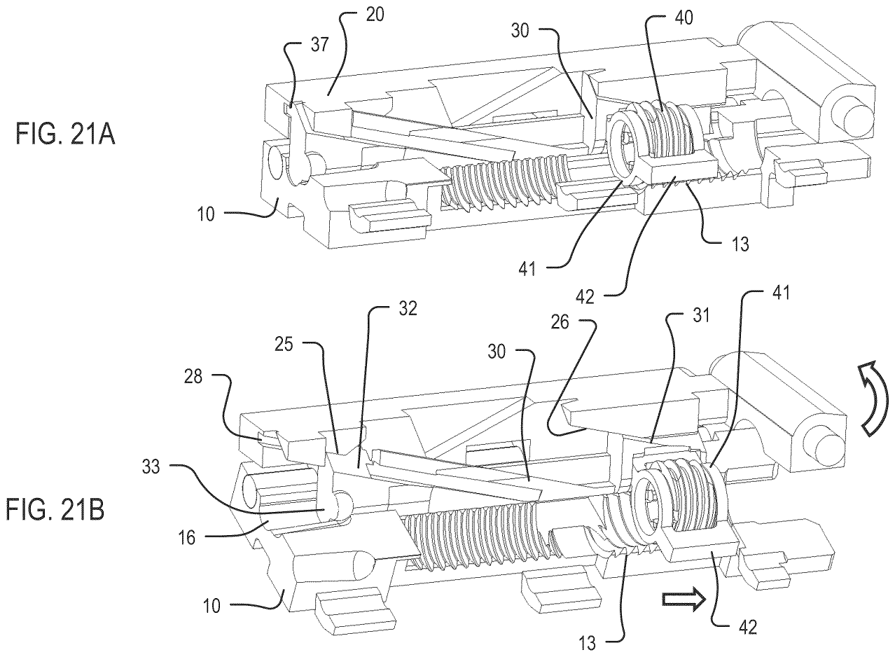

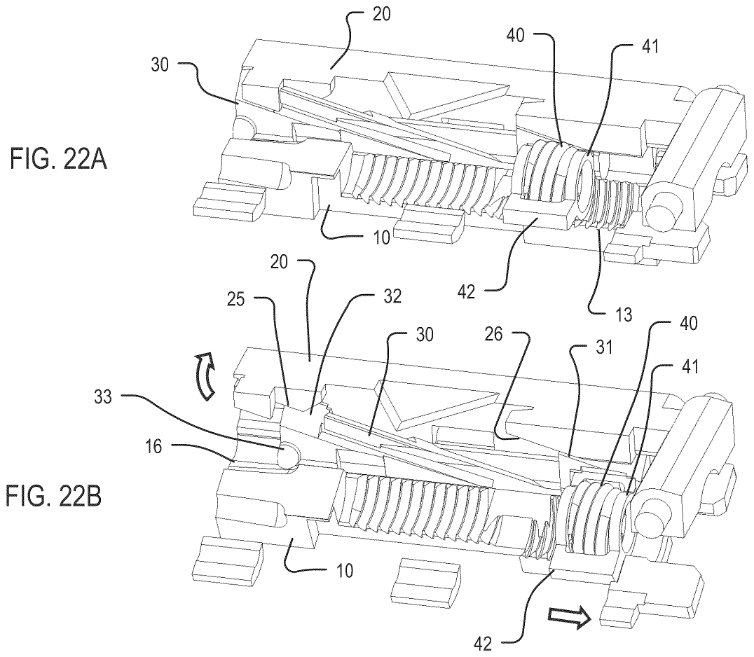

Referring now to FIGS. 20A-23 a method of distracting implant 100 will be disclosed. FIGS. 20A, 20B, 21A, and 21B are partial parts section views of implant 100 from a distal perspective. FIGS. 20A, 21A, and 22A show implant 100 in a collapsed position before actuation of first set screw 40 and FIGS. 20B, 21B, and 22B show implant 100 in a distracted position after actuation of first set screw 40. The angulation wedges 55, second set screw 50, and surgical tool 200 are removed in FIGS. 21A, 21B, 22A, and 22B. FIGS. 22A and 22B are corresponding partial parts section views of FIGS. 21A and 22B, respectively, from a proximal perspective.

A surgeon may insert surgical tool 200 within the access aperture 17 of the proximal end 100P of implant 100 such that the drive end of tool 200 may rotate the first set screw 40. In doing so, first set screw 40 is translated linearly within track 13 thereby pulling first set screw frame 41 in the longitudinal direction. Because first set screw frame 41 is coupled to distraction wedges 30 via wing like protrusions 42 being nested within cavity 35 the distraction wedges 30 also move in the longitudinal direction towards the proximal end 100P of implant 100. As shown in FIGS. 20B, 21B, and 22B, each distraction wedge 30 pushes the adjacent arm 20 away. For example, the ramps 31, 32 of distraction wedge 30 act against the ramps 25, 26 of arm 20. In this way, the distraction wedge 30 causes the adjacent arm 20 to pivot away from the frame 10. Stated another way, the pair of distraction wedges 30 cause the frame 10 to rotate about the central rotation axis R1 (see FIG. 18) thereby distracting the superior and inferior endplates 1, 2.

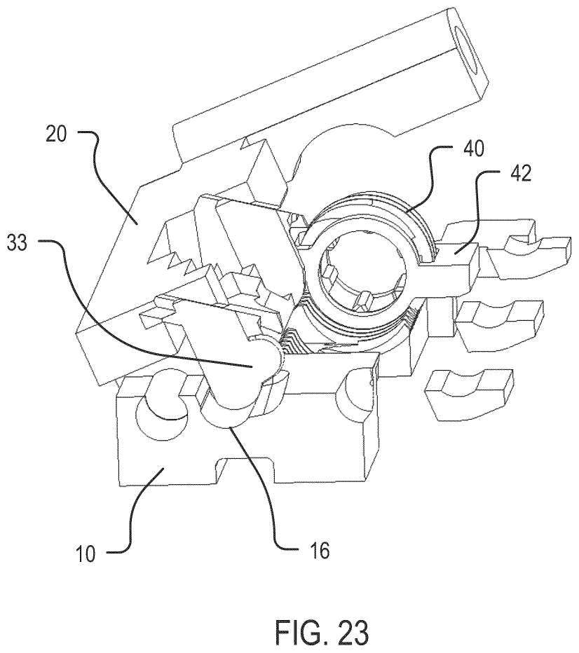

As seen best in FIG. 23, it is shown that as the frame 10 rotates relative to the arm 20, the distraction wedge 30 pivots in the circular channel 16. In this way, the distraction wedges 30 move forward and backward in the longitudinal direction while also rotating, at least partially, within the cylindrical channel 16 about a corresponding axis defined by the channel 16. The first set screw frame 41 may also rotate to accommodate this functionality.

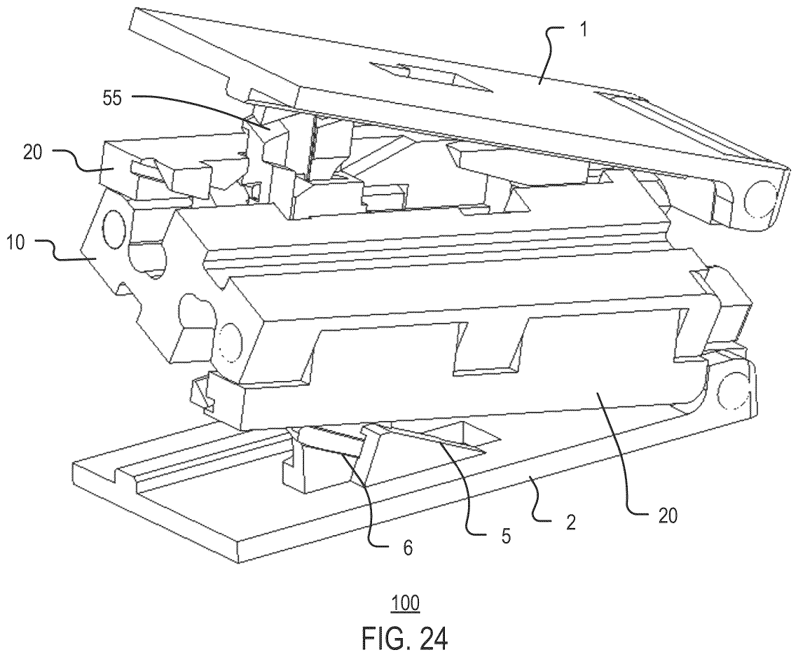

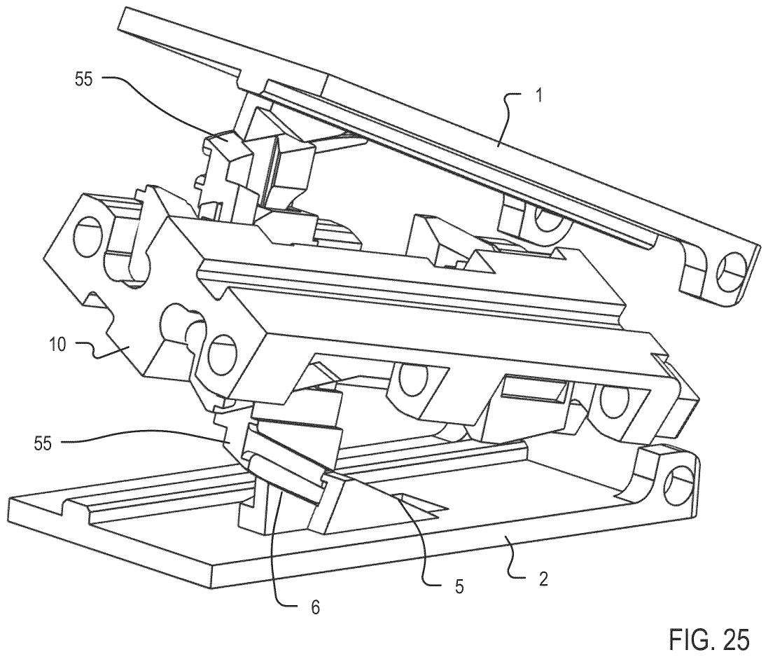

Referring generally to FIGS. 24-26, various views of implant 100 being positioned in a lordosed and distracted configuration are shown. In this particular configuration, implant 100 may be considered to be lordosed because an interior angle measured between an outside surface of superior endplate 1 and inferior endplate 2 is greater than a corresponding interior angle of implant 100 in the collapsed position. Additionally, implant 100 is distracted because a spacing distance between the superior endplate 1 and inferior endplate 2 is increased relative to the collapsed position. However, the distraction is not “parallel” on account of the lordotic angle between the superior endplate 1 and inferior endplate 2. As seen in FIG. 24, arms 20 are angulated outward by pivoting along the longitudinal pivoting axes L1, L2 (see FIG. 1A) which urges the superior and inferior endplates 1, 2 apart in the vertical direction. Additionally, the angulation wedges 55 are shown fully extended away from the arms 20 in the vertical direction thereby pivoting the superior and inferior endplates 1, 2 away from one another along the transverse pivoting axes T1, T2 (see FIG. 1B). As seen in FIG. 25, the arms 20 have been removed to shown how each of the angulation wedges 55 have moved along the ramps 5, 6 of endplates 1, 2 to thereby incline implant 100.

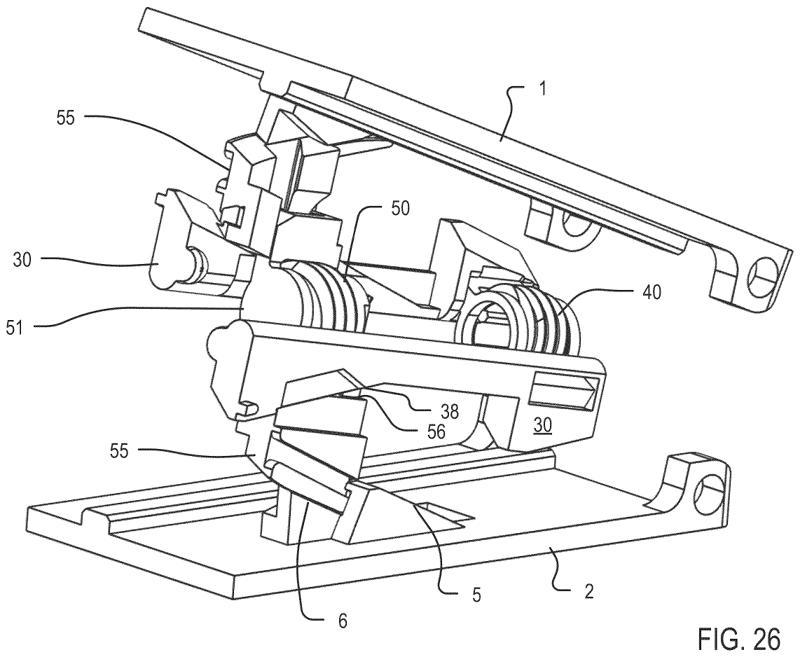

As seen in FIG. 26, the frame 10 has been removed to show how the angulation wedges 55 are also operably coupled with the distraction wedges 30. In this embodiment, the angulation wedges 55 also act against the ramped surfaces of the distraction wedges 30. For example, sliding surface 56 acts against ramped surface 38 of the distraction wedge 30. In this way, angulation wedge 55 pulls double duty in that it acts against two different inclined ramp surfaces which increases the relative lordotic angle adjustment vs. acting against a single inclined ramp surface.

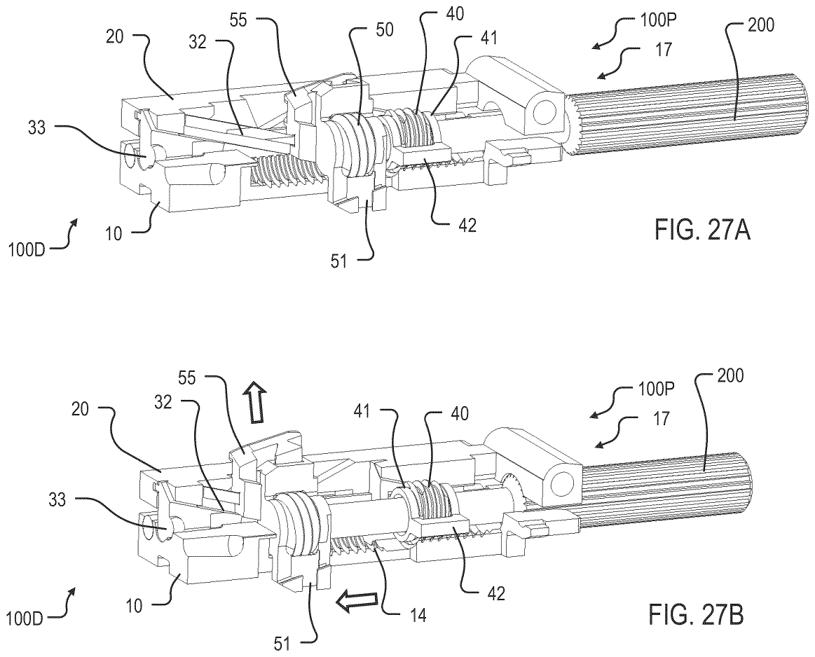

Referring generally to FIGS. 27A-30B a method of adjusting the lordosis of implant 100 will be discussed. FIGS. 27A-30B illustrate various partial parts section views showing how implant 100 may be moved from the collapsed configuration to a lordosed configuration by actuating a second set screw 50 via surgical tool 200 are illustrated.

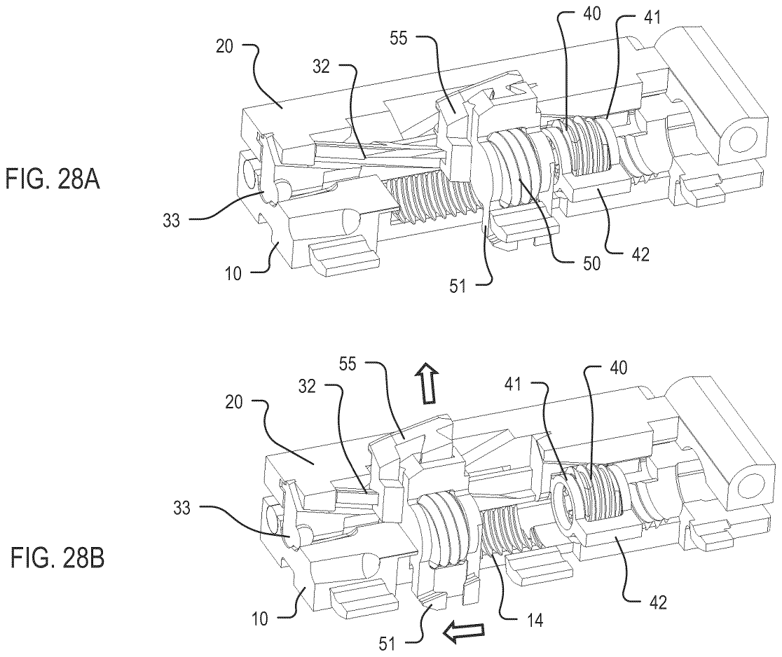

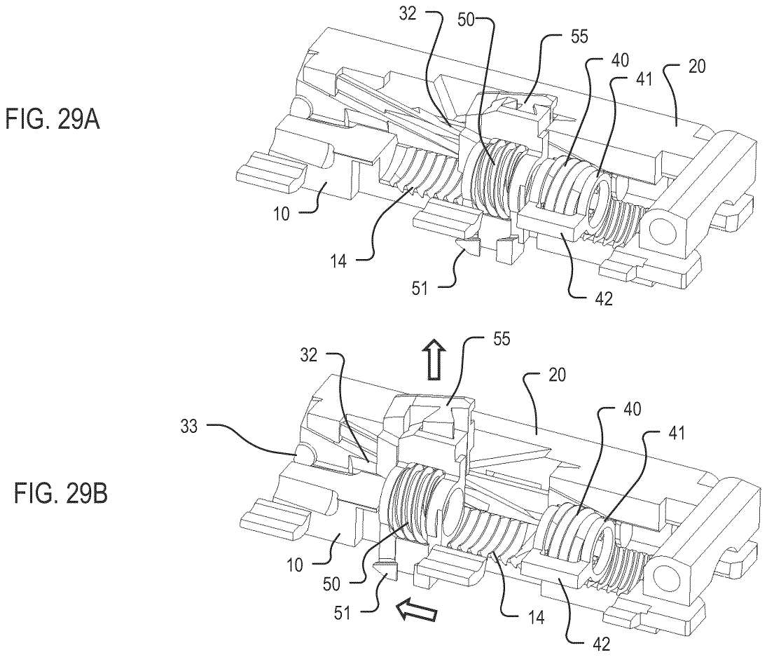

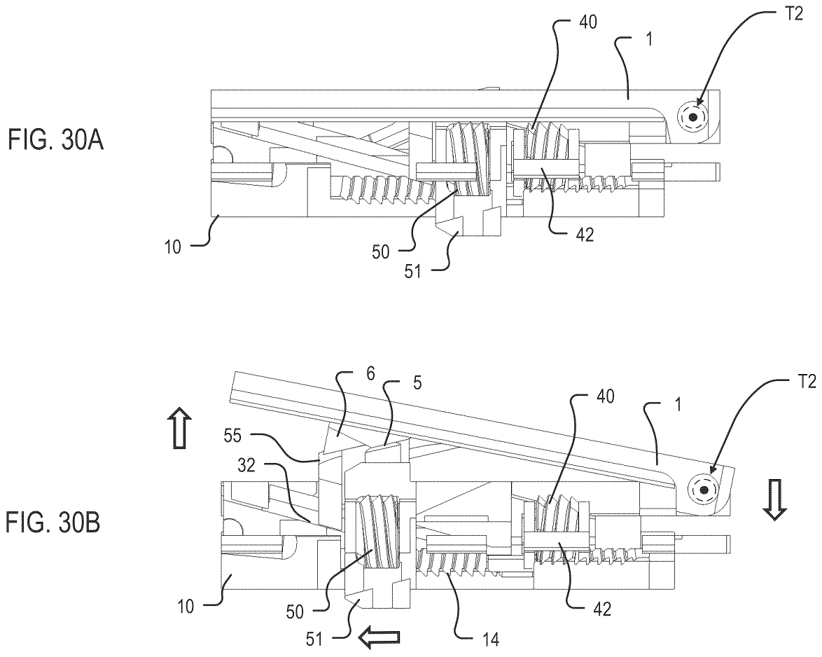

FIGS. 27A, 28A, 29A, and 30A illustrate implant 100 in a collapsed configuration. FIGS. 27B, 28B, 29B, and 30B illustrate implant 100 in a lordosed configuration. FIGS. 27A and 27B illustrate the surgical tool 200 interacting with second set screw 50 from a distal perspective. FIGS. 28A-30B illustrate the operation of angulation wedges 55 from a distal perspective and with the surgical tool 200 removed for ease of understanding. FIGS. 29A-29B illustrate the operation of angulation wedges 55 from a proximal perspective and with the surgical tool 200 removed for ease of understanding. FIGS. 30A and 30B are side section views showing the operation of distraction wedges.

As shown in FIGS. 27A-27B, a surgeon may insert surgical tool 200 within the access aperture 17 of the proximal end 100P of implant 100 and through the center of set screw 40 such that the drive end of tool 200 may rotate the second set screw 50. In doing so, only the second set screw 50 is translated linearly within track 14 thereby moving second set screw frame 51 in the longitudinal direction towards the distal end 100D. Because second set screw frame 51 is coupled to angulation wedges 55 via rail 53 and channel 52 (see FIG. 14) the angulation wedges 55 also move in the longitudinal direction towards the distal end 100D of implant 100. As explained in further detail below, and because of the particular mating arrangement between each corresponding angulation wedge 55, distraction wedge 30, and endplates 1, 2, the angulation wedge 55 may move away from second set screw frame 51 to cause the superior and inferior endplates 1, 2 to angulate.

As shown in FIGS. 27B, 28B, 29B and 30B, the angulation wedge 55 moves in the vertical direction away from second set screw frame 51. For example, angulation wedge 55 may ride along the ramp 32 of arm 20 on account of rail portion of ramp 32 being operably mated with channel 57. At the same time, angulation wedge 55 may ride along the ramps 5, 6 of the corresponding endplate 1, 2 thereby causing the superior and inferior endplates to become angled with respect to one another. With reference, to FIG. 30B, the pivoting of superior endplate 1 about transverse pivoting axis T2 is illustrated. As shown, angulation wedge 55 is linearly translated in the longitudinal direction and acts against ramp 32 of distraction wedge 30 and ramps 5 and 6 of superior endplate 1. In doing so, angulation wedge 55 urges superior endplate 1 upwards in the vertical direction thereby causing superior endplate 1 to pivot about the transverse pivoting axis T2. For example, the proximal side of superior endplate 1 may move downward in the vertical direction and the distal side of superior endplate 1 may move upward in the vertical direction. Although not illustrated in FIGS. 30A and 30B, it shall be appreciated that the angulation wedge 55 associated with the inferior endplate 2 may cause the same type of pivoting by the inferior endplate 2.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

What is claimed is:

1. An expandable implant pivotally movable between a collapsed position, an expanded position, comprising:
a first pin extending in a longitudinal direction and a second pin extending in a transverse direction;
a frame pivotally coupled to at least one arm, a superior endplate, and an inferior endplate, the frame having: a first track for rotatably supporting a first screw, and a second track for rotatably supporting a second screw, and the frame being configured to support the first pin;
the at least one arm being pivotally coupled to at least one of the superior endplate or the inferior endplate and being configured to support the first pin and the second pin;
at least one distraction wedge engaged with the at least one arm and the first screw, wherein rotation of the first screw is configured to cause the at least one distraction wedge to move in the longitudinal direction thereby rotating the frame about the first pin and urging the superior endplate and inferior endplate into a distracted configuration.

2. The expandable implant of claim 1, wherein an end of the frame comprises a chamfered edge disposed for pivotally rotating along a correspondingly curved socket of one of the superior endplate or inferior endplate.

3. The expandable implant of claim 1, further comprising:
at least one angulation wedge engaged with the at least one distraction wedge and the second screw,
wherein rotation of the second screw is configured to cause the at least one angulation wedge to move in the longitudinal direction thereby urging the superior endplate and the inferior endplate into an angled configuration.

4. The expandable implant of claim 3, wherein the at least one distraction wedge further comprises a ramp including at least one rail or channel configured to slidably mate with a corresponding ramp of the at least one angulation wedge.

5. The expandable implant of claim 3, wherein the at least one angulation wedge further comprises at least one ramp including at least one rail or channel configured to slidably mate with a corresponding ramp of one of the superior endplate or the inferior endplate.

6. The expandable implant of claim 3, further comprising:
a first screw case configured to surround the first screw and move with the first screw in the longitudinal direction of the frame and a second screw case configured to surround the second screw and move with the second screw in the longitudinal direction of the frame,
wherein the first screw case includes at least one boss configured for disposal in a corresponding eyelet of the at least one distraction wedge, and wherein the first screw case is rotatable with respect to the longitudinal direction of the frame; and
wherein the second screw case includes at least one groove configured to mate with a corresponding ramp of the at least one distraction wedge.

7. The expandable implant of claim 6, wherein the at least one groove of the second screw case is a dovetail groove.

8. The expandable implant of claim 1, wherein the at least one distraction wedge is engaged with at least one ramp of the at least one arm.

9. The expandable implant of claim 8, wherein the at least one arm includes at least one rail or channel configured to mate with the at least one distraction wedge.

10. The expandable implant of claim 1, wherein rotation of the first screw is configured to urge the superior endplate and inferior endplate into the distracted configuration be moving each of the superior endplate and inferior endplate away from the frame by an equal amount.

11. The expandable implant of claim 1, wherein the first screw and second screw are coaxially aligned and oppositely threaded.

12. An expandable implant pivotally movable into an angled position, comprising:
a frame operatively coupled to a first arm and a second arm on opposite sides of the frame, the first arm being operatively coupled to a superior endplate and the second arm being operatively coupled to an inferior endplate;
the frame having: a first channel for supporting a first pin extending in a longitudinal direction of the frame, a second channel for supporting a second pin extending in the longitudinal direction of the frame, and a track for supporting at least one actuator;
the first arm having: a third channel for supporting the first pin and a fourth channel for supporting a third pin extending in a transverse direction of the first arm, the superior endplate being pivotally coupled to the first arm via the third pin;
the second arm having: a fifth channel for supporting the second pin and a sixth channel for supporting a fourth pin extending in a transverse direction of the second arm, the superior endplate being pivotally coupled to the second arm via the fourth pin;
at least one wedge operatively coupled to the actuator and being configured to:
rotate the frame about the first and second pins thereby urging the superior endplate and inferior endplate into a distracted configuration; and
rotate the superior endplate and the inferior endplate about the third and fourth pins into an angled configuration.

13. The expandable implant of claim 12, wherein:
an end of the first arm comprises a chamfered edge disposed for pivotally rotating within a correspondingly curved socket of the superior endplate, and
an end of the second arm comprises a chamfered edge disposed for pivotally rotating within a correspondingly curved socket of the inferior endplate.

14. The expandable implant of claim 12, wherein the at least one actuator comprises a first screw and a second screw.

15. The expandable implant of claim 13, wherein the at least one wedge comprises:
a first distraction wedge and a second distraction wedge;
a first angulation wedge and a second angulation wedge;
the first distraction wedge further comprises a ramp including at least one rail or channel configured to slidably mate with a corresponding ramp of the first angulation wedge, and
the second distraction wedge further comprises a ramp including at least one rail or channel configured to slidably mate with a corresponding ramp of the second angulation wedge.

16. The expandable implant of claim 15, wherein:
the first angulation wedge further comprises at least one ramp including at least one rail or channel configured to slidably mate with a corresponding ramp of the superior endplate, and
the second angulation wedge further comprises at least one ramp including at least one rail or channel configured to slidably mate with a corresponding ramp of the inferior endplate.

17. The expandable implant of claim 16, further comprising:
a first screw case configured to surround the first screw and move with the first screw in the longitudinal direction of the frame;
a second screw case configured to surround the second screw and move with the second set screw in the longitudinal direction of the frame,
wherein the first screw case includes a first boss configured for disposal in a corresponding eyelet of the first distraction wedge and a second boss configured for disposal in a corresponding eyelet of the second distraction wedge, and the first screw case is rotatable with respect to the longitudinal direction of the frame, and
wherein the second screw case includes a first groove configured to mate with a corresponding ramp of the first distraction wedge and a second groove configured to mate with a corresponding ramp of the second distraction wedge.

18. The expandable implant of claim 16, wherein the first distraction wedge is engaged with at least one ramp of the first arm and the second distraction wedge is engaged with at least one ramp of the second arm.

19. The expandable implant of claim 18, wherein the first arm includes at least one rail or channel configured to mate with the first distraction wedge and the second arm includes at least one rail or channel configured to mate with the second distraction wedge.

20. The expandable implant of claim 16, wherein rotation of the first screw is configured to urge the superior endplate and inferior endplate into the distracted configuration be moving each of the superior endplate and inferior endplate away from the frame an equal amount.
